(19) United States
(12) Patent Application Publication
(10) Pub. No.: US 2011/0130844 A1
(43) Pub. Date: Jun. 2, 2011

(54) PYROLYTIC CARBON IMPLANT WITH ADHESIVE POLYMER OR ELASTOMER LAYER

(76) Inventors: Yves-Alain Ratron, Grenoble (FR); Michel Hassler, Saint Ismier (FR)

(21) Appl. No.: 12/933,873

(22) PCT Filed: Mar. 23, 2009
(86) PCT No.: PCT/EP09/53395

§ 371 Date: Oct. 26, 2010

(30) Foreign Application Priority Data
Mar. 21, 2008 (FR) 0851871
Mar. 21, 2008 (FR) 0851875

(51) Int. Cl. A61F 2/30; B29C 65/48

(52) U.S. Cl. 623/23.42; 156/155

(57) ABSTRACT

The invention relates to an articular resurfacing implant which is characterized in that it is composed of a sheet (1) of pyrolytic carbon deposited on a graphite substrate and a layer (8) composed of an adhesive elastomer or polymer material adherent to the inner surface of the sheet (1) on the substrate side. The adhesive elastomer or polymer material layer (8) allows the implant to be adherent to bone (3) and acts as a shock absorber layer. In one embodiment the implant further comprises a hard material, e.g. metallic, sheet (4).

The present invention relates to the field of arthroplasty, and more particularly to techniques for repairing articular (or joint) extremities.

Surgery to repair articular extremities is in a phase of rapid expansion by virtue of the lengthening of lifetime but also because numerous materials and proven surgical techniques are allowing a rapid improvement in the clinical pictures after the intervention and are making long-lasting immobilization of patients increasingly less necessary.

However, in spite of the development of numerous materials, articular prostheses are still not entirely satisfactory.

Metallic prostheses, for example, have numerous drawbacks, particularly owing to the mechanical stresses that they induce on the cartilaginous surfaces of the articular surfaces in contact with said metallic prostheses; these mechanical stresses may result in discomfort and pain, going as far as immobilization and a rapid deterioration in the clinical picture.

Indeed, despite the advances in the field of materials, and the innovations in tissue repair, there is no entirely satisfactory treatment and no material capable of providing a truly satisfactory substitute for the extraordinary properties of the articular cartilage.

Pyrolytic carbon has mechanical properties which are extremely advantageous for its use as an orthopaedic implant. Its elastic modulus is close to that of the cortical bone. Its use as an articular implant in the hand and as a coating on heart valves has established the fact it is ideally biocompatible, does not give rise to inflammatory reaction, allows effective biological fastening and does not give rise to complications. Moreover, cell growth on pyrolytic carbon is considered to be acceptable.

Its elastic modulus, of between 20 and 25 GPa for a density of between 1.7 and 2.0 g·cm³ (for bone, the respective values are 15 to 20 GPa and 2.0 g·cm³), eliminates the mechanical stresses and the necroses that are observed with metallic prostheses.

Pyrolytic carbon is obtained by thermal decomposition of gaseous hydrocarbons at high temperature by a process referred to as Chemical Vapour Deposition (CVD). Although in theory this allows deposition of virtually all metallic or non-metallic elements onto numerous substrates, it is graphite which possesses the most advantageous properties for deposition of pyrolytic carbon, and more particularly its coefficient of thermal expansion.

For producing implants with a coating of pyrolytic carbon, a graphite substrate is introduced into a chamber, which is heated at between 1200° C. and 1500° C., and then a hydrocarbon gas such as propane is introduced; the extreme temperature destroys the carbon-hydrogen bonds and allows carbon atoms to be deposited on the graphite substrate. In this way, layers of 300 to 600 microns are deposited on substrates. The physical and mechanical properties of the material obtained lie between those of graphite and those of diamond.

Pyrolytic carbon is used in orthopaedics, and more particularly in the surgery of the hand, for example, for the manufacture of implants such as interposition implants, scaphoid implants or carpometacarpal implants (U.S. Pat. No. 6,090,145). These implants have no mechanical or chemical fastening to the bone; they are stabilized by their positioning, and for those which possess a root or insertion rod in the long bone after insertion, the bony growth around the implant provides it with mechanical stabilization within a period of 6 to 24 months.

Also known, from US 2007/0225822, are metallic orthopaedic implants which comprise a surface coated with pyrolytic carbon.

Apart from the fact that metals are not ideal substrates for the CVD deposition of pyrolytic carbon, the application of pyrolytic carbon to metal means that the resulting, or overall, elastic modulus of the surface of the prosthesis will be increased by virtue of the presence of the metal, which is less elastic than the pyrolytic carbon. Consequently a pyrolytic carbon/metal composite does not retain all of the extremely advantageous properties of the pyrolytic carbon.

Also known are orthopaedic implants which are composed of a graphite substrate on which a layer of pyrolytic carbon is deposited (U.S. Pat. No. 6,090,145 and FR 2105998).

Another known problem which has been itemized is the shock resistance or proper absorption of mechanical or vibratory stresses: for example, those due to walking when the implant is positioned in a joint of the lower limbs, for example a hip or knee articulation, for example on the femoral condyles. When the implant is positioned in a joint of the upper limbs, the shocks may be due to the gripping of heavy objects or, for example, to the vibrations caused by means of transport.

U.S. Pat. No. 5,201,881 discloses metal prosthetic knee joints which comprise shock-absorbing means.

Apart from the fact that the prosthetic joints described are metallic joints, the solutions proposed entail translation of the polymeric parts relative to the metal parts, which are not necessarily possible with parts made of pyrolytic carbon, and particularly with pyrolytic carbon parts without a graphite substrate.

US 2005/0171604 discloses a prosthesis composed of an upper low-friction layer (material coated with pyrolytic carbon) and a damping layer (elastomer). The prosthesis is fixed to the bone by means of adhesives.

Moreover, FR 2105998 discloses articular implants which are composed of a part comprising a pyrolytic carbon surface deposited on a substrate and a layer of elastomer or polymer material, the layer of polymer material being fixed by an adhesive.

Interposition of a shock-absorbing material could be problematic for the adhesion to other parts of the prostheses and/or to the bone and especially for delamination risk between adhesive and shock-absorbing material under stresses applied on the prostheses

The present invention allows the various technical problems outlined above to be solved, by proposing the advantageous combination of the pyrolytic carbon properties and those of an adhesive polymer material or elastomer layer. By definition, this layer is preconstituted and for example it takes the form of a crosslinked or polymerized network. Mechanical and longevity properties are known or accessible since they depend on the choice of the material, its crosslinking or polymerization degree, its thickness, its adhesiveness, its elastic modulus. The person skilled in the art has at his disposal all kind of adhesive polymer or oligomer materials which can be used with an adapted thickness and elastic modulus. Thus, the present invention allows him to combine, in a synergetic way and in the best conditions, damping and adhesion. The present invention thus allows solving problems induced by mechanical and vibratory stresses on joint.

An object of the present invention is a resurfacing implant composed of a sheet of pyrolytic carbon and a layer composed of an adhesive elastomer or polymer material adherent to the sheet. The pyrolytic carbon surface forms the joint sliding surface. The adhesive layer is placed on the opposite side, i.e. the inner surface of the sheet with reference to its position when the implant is placed on bone or cartilage.

An other object of the invention is a resurfacing kit comprising on one hand said sheet, and on this other hand the adhesive material layer.

The fastening implant on cartilage or bone is directly ensured by the polymer or adhesive elastomer material. The invention allows the replacement shape for shape of all or some of the joint cartilage and optionally of part of the subjacent bone, i.e. the cartilage and/or part of the bone is replaced after surgical resection or the cartilage and/or part of bone is replaced having been previously destroyed by wear or the like. “Shape for shape” means that the shape of the inner surface of the implant, i.e. the surface facing the bone or cartilage, complements and matches, as closely as possible, the shape of the surface on which the implant will be placed. In an embodiment, shape for shape means further that the implant has also the volume of the bone or cartilage to be replaced.

In such an implant, the pyrolytic carbon sheet is a low thickness piece, designed to be closely adapted to the cartilage or bone surface to be resurfaced. In one embodiment the implant or the piece has the form of dome, especially a spherical dome.

According to one embodiment, the resurfacing implant is composed of a pyrolytic carbon sheet having no substrate constituting the support, the adhesive polymer or elastomer material layer being directly adherent to the inner surface of the pyrolytic carbon sheet. In this embodiment the implant has a thickness of less than 2 mm and in one embodiment of between 0.5 and 1.5 mm.

In an other embodiment, the resurfacing implant is composed of a pyrolytic carbon sheet deposited on a graphite substrate, and has a thickness of less than 4 mm, preferably of between 1.5 and 3.5 mm.

In one embodiment, following deposition of the pyrolytic carbon, the resulting resurfacing implant is machined and hollowed out in such a way as to remove at least some of the lower layer of pyrolytic carbon and graphite, to give an implant composed of pyrolytic carbon over a low thickness of graphite, or solely of a sheet of pyrolytic carbon. In this embodiment the implant has a thickness of less than 2 mm and preferably of between 0.5 and 1.5 mm.

The elastomer or polymer material layer is a layer which allows shock absorption to take place; i.e., it comprises and/or consists of a material which allows the load and/or the pressure to be distributed over the bone tissue, which is subjected to various, more or less complex stresses, both static and dynamic, resulting from flexion, traction, rotation and compression movements.

By adhesive polymer or elastomer material is meant an adhesive material selected among silicone rubbers, polyurethanes, polyester urethane copolymers and polyether polyester copolymers.

In one embodiment said layer of elastomer or polymer material has a thickness of between 0.2 and 3 mm, preferably between 0.5 and 3 mm

In one particular embodiment the shock-absorbing adhesive material is a rubber or other silicone material, for example an adhesive silicon elastomer.

In an other embodiment of the present invention, the implant comprises a pyrolytic carbon sheet with or without substrate, an adhesive elastomer or polymer material layer and a hard material, e.g. metallic, sheet, with the adhesive elastomer or polymer material layer sandwiched between the carbon and the hard material sheets, wherein the latter sheet is intended to adhere to the bone or cartilage surface.

According to an advantageous characteristic, the implant comprising the hard material, e.g. metallic, sheet has a stiffness comprised between 150 and 800 daN/mm. To obtain this stiffness, the person skilled in the art may adapt the thickness of the carbon and hard material, e.g. metallic, sheets, and that of the adhesive oligomer or polymer layer as well.

The hard material, e.g. metallic, sheet may have a thickness equal or above 1.5 mm. The hard material sheet may be titanium alloys, chromium-cobalt alloys, stainless steel, hard polymers, ceramics, memory-shape materials, etc.

In this embodiment the implant comprising a hard material, e.g. metallic, sheet can adheres to the bone or cartilage by means of bioresorbable adhesive, such as biocompatible cement, biological glue or polymeric glue such as fibrin.

It will be possible to use osteoinductive biological cements with or without tissue extracts, in order to promote bone colonization.

The biological cements are, for example, bioabsorbable cements such as calcium phosphate cements which are biocompatible and osteoinductive. Their high biocompatibility allows the incorporation of pharmaceutical active principles and of living cells in the form of tissue extracts.

The tissue extracts are living biological tissue extracts or autologous, allogenic or xenogenic cells. These cells will preferably be selected from those capable of stimulating the regeneration of cartilaginous tissues, for example chondrocytes, which are isolated and multiplied by techniques known to a person skilled in the art, such as cell culture from cartilage biopsy.

In one embodiment the tissue extracts are selected from autologous, allogenic or xenogenic cells which belong to the line of chondrocytes or chondrocyte progenitor cells.

In one embodiment the implant adheres to the bone extremity by polymeric adhesives such as PMMAs.

The invention likewise provides the method of manufacturing an implant according to the invention, characterized in that it comprises the steps of:
a) providing a substrate,
b) depositing pyrolytic carbon on said substrate.

In one embodiment the substrate is made of graphite.

In one embodiment step b) is followed by a step b′) of totally or partly removing the substrate.

In another embodiment the method of manufacturing an implant according to the invention is characterized in that it comprises steps of:
a) providing a substrate,
b) depositing pyrolytic carbon on said substrate,
c) depositing a layer of adhesive elastomer or polymer material at the surface of the piece obtained, the adhesive properties of said layer allowing the implant to be fastened to bone.

In one embodiment the substrate is made of graphite.

In one embodiment step b) is followed by a step b′) of totally or partly removing the substrate.
In one embodiment said layer of elastomer or polymer material is a layer of material based on silicone.

In another embodiment the method of manufacturing an implant according to the invention is characterized in that it comprises steps of:
a) providing a substrate,
b) depositing pyrolytic carbon on said substrate,
c) depositing a layer of adhesive elastomer or polymer material at the surface of the piece obtained, and
depositing a hard material, e.g. metallic, sheet on the adhesive layer.

In one embodiment the substrate is made of graphite.

In one embodiment step b) is followed by a step b′) of totally or partly removing the substrate.

In one embodiment said layer of elastomer or polymer material is a layer of material based on silicone.

The hard material, e.g. metallic, sheet can adheres to the bone or cartilage surface by means of biocompatible adhesive, such as bioresorbable cement, biological glue or polymeric glue as described above.

The present invention also relates to a process for resurfacing cartilage in which an implant according to the invention is set on a bone or cartilage surface.

The process for resurfacing cartilage allows to replace, shape for shape, all or some of the joint cartilage and optionally of part of the subjacent bone, wherein the implant is composed of a pyrolytic carbon sheet having or not a substrate constituting the support and an adhesive polymer or elastomer material layer.

In one embodiment, the implant further comprises a hard material, e.g. metallic, sheet in such a way that the adhesive layer is sandwiched between the pyrolytic carbon sheet and the hard material, e.g. metallic, sheet.

The hard material, e.g. metallic, sheet can adheres to the bone or cartilage surface by means of a biocompatible adhesive, such as bioresorbable cement, biological glue or polymeric glue as described above.

Depending on the damage and wear of the articular cartilage, the implant is arranged in place after partial or complete cartilage resection.

In another embodiment of the process, the implant is arranged in place after complete cartilage resection and after resection of the subjacent bone to a specific depth.

In one embodiment the implant is placed on an articular bone head.

In an other embodiment, the implant is placed in a glenoid cavity.

The invention also relates to a resurfacing kit characterised in that it comprises a pyrolytic carbon sheet (1) and an adhesive elastomer or polymer material layer to be adherent on the sheet, sheet and layer being as described in any of the previous claims.

In one embodiment the resurfacing kit further comprises a hard material, e.g. metallic, sheet. This sheet is intended to be used in such a way that the adhesive layer is at the time of use sandwiched between the pyrolytic carbon sheet and the hard material, e.g. metallic, sheet, and that the latter sheet is adhered to the bone or cartilage surface.

Before adhesion to its substrate, the adhesive layer may be protected by a sheet of material that does not firmly adhere to the layer.

The invention will be appreciated more effectively in the light of the embodiments which are illustrated in the figures, which are diagrammatic representations of various embodiments by way of non-limiting example.

As an example of the implementation of the implants according to the invention is represented by the shoulder joint, or gleno-humeral joint, in which the bone structures are composed of two principal bones which engage: the humerus, whose upper joint part has a partially spherical, rounded shape, and the scapula, whose joint part or glenoid fossa articulates with the spherical part of the humerus and has a bowl shape.

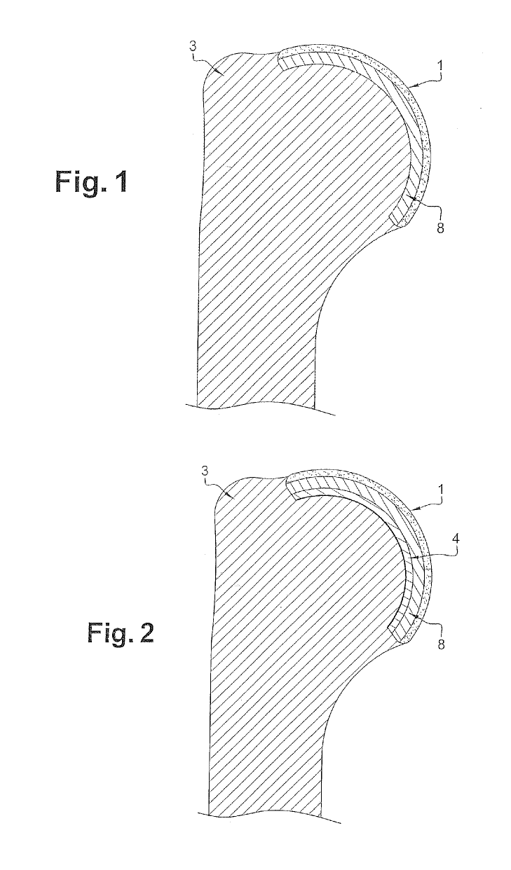

FIG. 1 shows a section through the head of the humerus which forms part of the articulation of the shoulder, carrying a resurfacing implant comprising a shock-absorbing layer and a pyrolytic carbon sheet according to the invention.

FIG. 2 shows a section through the head of the humerus which forms part of the articulation of the shoulder, carrying a resurfacing implant comprising a shock-absorbing layer sandwiched between a pyrolytic carbon sheet and a metallic sheet.

The implant shown in FIG. 1 is composed of a sheet of pyrolytic carbon (1) and an adhesive silicone layer (8). Said implant is fixed to the head, in this case the humeral head (3), by the self-adhesive silicone layer (8). The same property is used for the adhesion between the carbon sheet and the silicon layer.

The implant shown in FIG. 2 is composed of an adhesive silicone layer (8) sandwiched between a pyrolytic carbon sheet (1) and a metallic sheet (4). Said metallic sheet is fixed to the cartilage or bone surface (3) through a cement.

1-22. (canceled)

23. An implant for articular resurfacing, the implant comprising:
a sheet of pyrolytic carbon; and
a layer adherent to the sheet, the layer constituted of adhesive polymer or elastomer material.

24. The implant of claim 23, wherein the sheet of pyrolytic carbon is devoid of any substrate, the layer being adherent to an inner surface of the sheet of pyrolytic carbon.

25. The implant of claim 23, wherein the sheet has a thickness of less than 4 mm.

26. The implant of claim 25, wherein the sheet has a thickness between 1.5 mm and 3.5 mm.

27. The implant of claim 24, wherein the implant has a thickness of less than 2 mm.

28. The implant of claim 27, wherein the implant has a thickness between 0.5 mm and 1.5 mm.

29. The implant of claim 23, wherein the layer has a thickness between 0.2 mm and 3 mm.

30. The implant of claim 29, wherein the layer has a thickness between 0.5 mm and 3 mm.

31. The implant of claim 23, wherein the adhesive polymer or elastomer material is a material selected from the group consisting of: silicone rubbers, polyurethanes, polyester urethane copolymers, and polyether polyester copolymers.

32. The implant of claim 23, wherein the implant has a form adapted for placement on an articular bone head.

33. The implant of claim 23, wherein the implant has a form adapted for placement in a glenoid cavity.

34. The implant of claim 23, further comprising a sheet of hard material, wherein the layer is sandwiched between the sheet of pyrolytic carbon and the sheet of hard material.

35. The implant of claim 34, wherein the hard material is metal.

36. The implant of claim 34, wherein the implant has a stiffness between 150 daN/mm to 800 daN/mm.

37. The implant of claim 36, wherein the hard material is a material selected from the group consisting of: titanium alloy, chromium-cobalt alloy, stainless steel, hard polymer, and ceramic or memory-shape material.

38. A method of manufacturing an articular implant, comprising:
depositing pyrolytic carbon on all or part of a substrate to form a sheet of pyrolytic carbon.

39. The method of claim 38, further comprising:
depositing a layer of adhesive polymer or polymer material at a surface of the sheet of pyrolytic carbon, the adhesive properties of the layer allowing the implant to be fastened to bone.

40. The method of claim 39, further comprising:
depositing a hard material sheet onto the layer of adhesive polymer or polymer material.

41. The method of claim 40, wherein the hard material sheet is metallic.

42. The method of claim 38, wherein the substrate is pyrolytic carbon.

43. The method of claim 38, further comprising removing the substrate.

44. The method of claim 39, further comprising removing the substrate before depositing the layer of adhesive polymer or polymer material.

45. A process for resurfacing cartilage, the process comprising:
replacing, shape for shape, at least a portion of articular cartilage, or articular cartilage and subjacent bone, with an implant comprising a pyrolytic carbon sheet and a layer of adhesive polymer or elastomer material.

46. The process of claim 45, wherein the implant further comprises a sheet of hard material, wherein the adhesive layer is sandwiched between the pyrolytic carbon sheet and the sheet of hard material, and wherein the metallic sheet is adapted to adhere to a surface of the articular cartilage, or to a surface of the articular cartilage and the subjacent bone.

47. The process of claim 46, wherein the hard material is metal.

48. The process of claim 45, further comprising partially or completely resecting the articular cartilage before replacing the articular cartilage with the implant.

49. The process of claim 45, further comprising resecting the articular cartilage and resecting the subjacent bone to a specific depth before replacing the articular cartilage with the implant.

50. A resurfacing kit, the resurfacing kit comprising:
a pyrolytic carbon sheet, and a layer of adhesive elastomer or polymer material.

51. The resurfacing kit of claim 50, further comprising a sheet formed of a hard material sheet.

52. The resurfacing kit of claim 51, wherein the hard material sheet is a metallic sheet.
